SEQUENCE LISTING (28 sequences)

SEQ ID NO: 1 - DNA, 25 bp, synthetic construct
  source 1..25: mol_type = other DNA
ccaaacctgtaggccgactctgcaa

SEQ ID NO: 2 - DNA, 25 bp, synthetic construct
  source 1..25: mol_type = other DNA
ttgtgcaacgggacaatgctaggac

SEQ ID NO: 3 - DNA, 43 bp, synthetic construct
  source 1..43: mol_type = other DNA
atcgacttccgagacatcatcgacgtcaccgaactgcgggtgt

SEQ ID NO: 4 - DNA, 39 bp, synthetic construct
  source 1..39: mol_type = other DNA
gagtgcgtccaggcagagcgactccatccgcccccatgt

SEQ ID NO: 5 - DNA, 40 bp, synthetic construct
  source 1..40: mol_type = other DNA
gagtgcgtccaggcagagcgacttttaattcgctccttaa

SEQ ID NO: 6 - DNA, 45 bp, synthetic construct
  source 1..45: mol_type = other DNA
caagttagtttctcctattttacgacgtcaccgaactgcgggtgt

SEQ ID NO: 7 - DNA, 62 bp, synthetic construct
  source 1..62: mol_type = other DNA
gagccaaggggacgtatgaccttagcagacattgaacggcgacgtcaccgaactgcgggtgt

SEQ ID NO: 8 - DNA, 64 bp, synthetic construct
  source 1..64: mol_type = other DNA
gagtgcgtccaggcagagcgactctctggaagagggaactctgccctttaacttggctgaggcc

SEQ ID NO: 9 - DNA, 23 bp, synthetic construct
  source 1..23: mol_type = other DNA
agtcgctctgcctggacgcactc

SEQ ID NO: 10 - DNA, 23 bp, synthetic construct
  source 1..23: mol_type = other DNA
acacccgcagttcggtgacgtcg

SEQ ID NO: 11 - DNA, 20 bp, synthetic construct
  source 1..20: mol_type = other DNA
tacgcacaaccgaagcgtag

SEQ ID NO: 12 - DNA, 43 bp, synthetic construct
  source 1..43: mol_type = other DNA
acacccgcagttcggtgacgtcgaccggacagagcccattaca

SEQ ID NO: 13 - DNA, 44 bp, synthetic construct
  source 1..44: mol_type = other DNA
gagtgcgtccaggcagagcgactgcccattacaatattgtaacc

SEQ ID NO: 14 - DNA, 20 bp, synthetic construct
  source 1..20: mol_type = other DNA
ttggagtcgttcctgtcgtg

SEQ ID NO: 15 - DNA, 43 bp, synthetic construct
  source 1..43: mol_type = other DNA
acacccgcagttcggtgacgtcgtcacaacatagctgggcact

SEQ ID NO: 16 - DNA, 45 bp, synthetic construct
  source 1..45: mol_type = other DNA
gagtgcgtccaggcagagcgacttagctgggcactatagaggcca

SEQ ID NO: 17 - DNA, 20 bp, synthetic construct
  source 1..20: mol_type = other DNA
acctcagatcgctgcaaagt

SEQ ID NO: 18 - DNA, 43 bp, synthetic construct
  source 1..43: mol_type = other DNA
acacccgcagttcggtgacgtcggtgtcaggcgttggagacat

SEQ ID NO: 19 - DNA, 37 bp, synthetic construct
  source 1..37: mol_type = other DNA
gagtgcgtccaggcagagcgactttggagacatctgt

SEQ ID NO: 20 - DNA, 20 bp, synthetic construct
  source 1..20: mol_type = other DNA
gcattccacgcactgtagtt

SEQ ID NO: 21 - DNA, 47 bp, synthetic construct
  source 1..47: mol_type = other DNA
ctgccacgatcgactcgtctgcagtcattgcatgatttgtgccaagc

SEQ ID NO: 22 - DNA, 49 bp, synthetic construct
  source 1..49: mol_type = other DNA
gctggacagtgtgcggcagtcacaatttgtgccaagcattggagacaac

SEQ ID NO: 23 - DNA, 21 bp, synthetic construct
  source 1..21: mol_type = other DNA
catgctatgcaacgtcctgtc

SEQ ID NO: 24 - DNA, 46 bp, synthetic construct
  source 1..46: mol_type = other DNA
ctgccacgatcgactcgtctgcagtgtcaaagaccgttgtgtccag

SEQ ID NO: 25 - DNA, 45 bp, synthetic construct
  source 1..45: mol_type = other DNA
gctggacagtgtgcggcagtcacagtccagaagaaaaacaaagac

SEQ ID NO: 26 - DNA, 20 bp, synthetic construct
  source 1..20: mol_type = other DNA
actgacctcccacattccct

SEQ ID NO: 27 - DNA, 45 bp, synthetic construct
  source 1..45: mol_type = other DNA
ctgccacgatcgactcgtctgcagtgggccttgagcatctggatt

SEQ ID NO: 28 - DNA, 37 bp, synthetic construct
  source 1..37: mol_type = other DNA
gctggacagtgtgcggcagtcacaatctggattctgc

(19) United States
(12) Patent Application Publication
(10) Pub. No.: US 2024/0309430 A1
(43) Pub. Date: Sep. 19, 2024

(54) UNIVERSAL COMPOSITIONS AND METHODS FOR MULTIPLEX GENOTYPING

(71) Applicant: The Hong Kong University of Science and Technology, Hong Kong (CN)

(72) Inventors: I-Ming HSING, Hong Kong (CN); Xinyu ZHUANG, Hong Kong (CN)

(21) Appl. No.: 18/608,081

(22) Filed: Mar. 18, 2024

Related U.S. Application Data
(60) Provisional application No. 63/490,771, filed on Mar. 16, 2023.

(51) Int. Cl. C12Q 1/6818; C12Q 1/6844

(52) U.S. Cl. CPC C12Q 1/6818; C12Q 1/6844

(57) ABSTRACT

The subject invention pertains to the multiplex detection of nucleic acid molecules. This invention provides oligonucleotides labeled with a fluorophore and a quencher and oligonucleotides with target binding regions that are broadly compatible with the detection of multiple variant locus or multiple nucleic acid samples. Additionally, this disclosure also describes methods to label detection targets by asymmetric labeled amplification and differentiate the target sequences.

### CROSS-REFERENCE TO RELATED APPLICATION

This application claims the benefit of U.S. Provisional Application Ser. No. 63/490,771, filed Mar. 16, 2023, which is hereby incorporated by reference in its entirety including any tables, figures, or drawings.

### SEQUENCE LISTING

The Sequence Listing for this application is labeled “SeqList-as filed.xml” which was created on Mar. 5, 2024 and is 26,054 bytes. The entire contents of the sequence listing is incorporated herein by reference in its entirety.

### BACKGROUND OF THE INVENTION

Multiplexed RT-PCR is low cost and has high accessibility, usually achieved by incorporating several pairs of amplification primers and detection reporter probes [9]. The testing capacity per reaction of conventional RT-PCR is the key issue because the fluorescence channels of PCR thermocycler are limited at 5 to 6. Alternatively, PCR amplification combined with probe-based melting curve analysis solved[10] the throughput problems because the capacity can be increased by designing probes with different melting temperatures and different fluorescence modifications. It still has limitations, including the increased complexity of the system, modification costs, and high requirements for probe design. In addition, high-throughput genotyping assays can be done using microarray technology[11], in which hundreds to thousands of different probes are immobilized on a gene chip and fluorescently labeled. This oligonucleotide probe hybridization method involves the use of a rationally designed sequence to interrogate a specific mutation site and the fluorescent readout can determine which mutations are present in the sample. This technique requires specialized equipment and software to process the raw fluorescence data from the gene chip and has a high fabrication cost for chips[12]. Current strategies have moved towards sequencing-based techniques[ 13] due to their greater versatility. Instead of having a customized chip for every set of mutations to be tested, DNA sequencing can theoretically be used to identify mutations across the entire genome. A reverse transcription step can also be incorporated to determine gene expression profiles through RNA sequencing[ 14]. However, these powerful techniques are expensive, time-consuming, and laborious to apply to genetic disease screening. Usually, only when the subject is positive in the first-tier screening will a second-tier confirmatory test be performed. First-tier screening methods must be cheap, fast, and highly multiplexed because the main objective is to flag subjects who are suspected of having genetic diseases from a large pool of samples. Therefore, a rationally designed oligonucleotide probe-based approach is a good candidate method for this purpose.

The current gold standard method for multiplex genotyping is real-time Polymerase Chain Reaction (PCR)[15] in one close-tube reaction. Multiplex PCR assay by TaqMan (Roche Molecular Systems, Inc., Branchburg, New Jersey, USA) hydrolysis probes has been widely applied in the detection of different nucleic acid specimens[9], but the testing capacity in one reaction is limited because one TaqMan probe can only differentiate one target and typically only 5 to 6 fluorophore channels are supported by the commercial qPCR machines. Instead, probe-

based high-resolution melting (HRM) curve analysis[ 16] is a powerful tool for the identification of gene mutations or multiple nucleic acid sequences due to its high throughput, which allows differentiation among PCR amplicons that differ by as little as one base pair. The testing capacity is usually enlarged by designing the different lengths of probes with different melting temperatures (Tₘ) in up to 6 fluorescent channels. Dual-labeled TaqMan probe[ 17] is the most commonly utilized reporter system in the current commercial kits of melting curve assay. After generation of single-strand amplificons through asymmetric PCR, the fluorophore and quencher labeled probes were able to detect up to 15 respiratory pathogens[18]. Nevertheless, there still exist some limitations in the multiplex melting sensing process. Due to the requirement of one TaqMan probe for one target sequence[ 19], the design of reporter probes is still very time-consuming and increases the cost of fluorescence and quencher modification. Additionally, the increase of fluorogenic probes required will result in an increase in fluorescence background and a decrease in testing sensitivity[20].

Therefore, universal compositions and methods that are applicable to the detection of multiple variant locus or multiple nucleic acid samples are needed to address the abovementioned issues.

### BRIEF SUMMARY OF THE INVENTION

The invention pertains to the detection of genetic variants. The invention provides primers and probes and methods of detecting genetic variants by nucleic acid hybridization, specifically by measuring the fluorescence of fluorophore-labelled probes when bound to a target nucleotide sequence and in the presence of a quencher-labelled probe and when unbound from a target nucleotide sequence and in the absence of a quencher-labelled probe.

In certain embodiments, the subject methods can be used to detect genetic variations in infectious agents (e.g., bacteria or viruses) or genetic variations that can cause, or are associated with, diseases can be detected, including diseases indicated by single nucleotide polymorphisms (SNPs).

The invention also relates to these primers and probes, as well as to pharmaceutical compositions, to biological compositions, to detection kits, and to diagnostic kits.

### BRIEF DESCRIPTION OF THE DRAWINGS

FIGS. 1A-1B. The sensing principle of two designs of melting reporting system. FIG. 1A X-shaped compositions. During the melting sensing process, the fluorescence intensity is low at low temperatures and the fluorescence signal increases sharply when the reaction temperature reaches the Tm of the MI strand-target hybrid. FIG. 1B Y-shaped compositions. When the environmental temperature reaches the Tₘ of M1′ strand-target hybrid, the fluorescence intensity will decrease due to the FRET quenching effect.

FIGS. 2A-2B. The melting sensing process for detection of multiple mutation sites by two types of universal compositions. One fluorescence channel is capable of detecting three mutation loci. RFU refers to relative fluorescence unit and −d RFU/d T indicates the change rate of fluorescence signal. FIG. 2A Detection using X-shaped compositions. If the target sequences exist, melting plots will show downward peaks with the corresponding designed Tₘ value of the M strand. The Tₘ shift compared with wild-type sequences indicates the existence of mutation points. FIG. 2B Detection using Y-shaped compositions. The fluorescence signal change is opposite to that of the X-shaped compositions. Upward melting peaks can be correlated to the existence of mutation sites.

FIGS. 3A-3B. The applications for detection of multiple nucleic acid molecules using two designs of universal compositions. One fluorescence channel can identify at least six subtypes. −d RFU/d T-T records the change rate of the fluorescence signal with the increase of temperature. FIG. 3A Detection using X-shaped compositions. If the reaction has six targets, the result will have six upward melting peaks at their pre-designed Tₘ values. FIG. 3B Detection using Y-shaped compositions. Six peaks with differentiable Tₘ values will be observed in melting plots. The testing capacity can be further expanded by increasing the number of fluorescence channels.

FIG. 4. The working principle of incorporation of asymmetric labeled amplification and melting sensing by universal probe composition. The target sequence will be first amplified and labeled by an excess amount of PCR forward primer and labeled reverse primer. After asymmetric amplification, a single strand labeled target sequence will be generated for further melting curve analysis. The universal probe compositions include one MI strand and one set of F-Probe and Q-Probe, which will be used as reporting system for the melting sensing process. The complementary sequence of the universal region will fully bind with the Q-Probe. Unmodified M1 strand will bind with F-Probe and target sequence.

FIG. 5. The melting process for detection of multiple labeled target sequences. At least six samples can be detected simultaneously in one fluorescence channel. Each target will be assigned one target-specific M1 strand. One universal set of F-Probe and Q-Probe will be shared among all six targets. All targets in the reaction are labeled with a universal region through asymmetric labeling amplification at the beginning. By conducting a melting curve analysis, six downward peaks will appear when all six targets exist in this reaction.

FIGS. 6A-6B. Melting curve results of one mutation site (SNP) detected in the FAM channel using FIG. 6A X-shaped compositions. FIG. 6B Y-shaped compositions. The wild type means the template doesn't have this mutation while the mutant type has a single base mutation site. NTC represents no template control. The 5° C. left shift in melting peaks indicates the existence of the mutation site.

FIGS. 7A-7D. Detection results of the template with three possible mutation sites using the X-shaped structure. Each Tₘ value of the peak represents the mutation status of target sites. FIG. 7A Homozygous mutant-type target. It will have three distinguishable melting peaks. FIG. 7B Homozygous wild-type target. It will have three distinguishable melting peaks. FIG. 7C Heterozygous wild-type and mutant-type targets. It will have six distinguishable melting peaks. FIG. 7D Melting curve combo. The combo is the superimposition of the curves to show the difference in Tₘ.

FIGS. 8A-8C. Barcoding amplification and X-shaped compositions were incorporated for the detection of HPV subtypes. The size distribution results in FIG. 8A show the products from barcoding PCR are longer than that of normal PCR. The melting curve results in FIG. 8B show that all five subtypes can be correctly identified in FAM and HEX channels. FIG. 8C shows that no cross-reactivity was observed.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

The terms “label,” “detectable label, “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a fluorescent label into the peptide or used to detect antibodies specifically reactive with the peptide. The term includes combinations of single labeling agents, e.g., a combination of fluorophores that provides a unique detectable signature, e.g., at a particular wavelength or combination of wavelengths. In the context of detecting nucleic acids (e.g., target sequences), the probes can, typically, be labeled with radioisotopes, fluorescent labels (fluorophores), or luminescent agents.

As used herein, the term “positive,” when referring to a result or signal, indicates the presence of an analyte or item that is being detected in a sample. The term “negative,” when referring to a result or signal, indicates the absence of an analyte or item that is being detected in a sample. Positive and negative are typically determined by comparison to at least one control, e.g., a threshold level that is required for a sample to be determined positive, or a negative control (e.g., a known blank). A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters, and will understand which controls are valuable in a given situation and be able to analyze data based on comparisons to control values. Controls are also valuable for determining the significance of data. For example, if values for a given parameter are variable in controls, variation in test samples will not be considered as significant.

The term “sample” encompasses a variety of sample types containing a nucleotide. The term encompasses bodily fluids such as blood, blood components, saliva, nasal mucous, serum, plasma, cerebrospinal fluid (CSF), urine and other liquid samples of biological origin, solid tissue biopsy, tissue cultures, or supernatant taken from cultured patient cells. A sample further encompasses samples from the environment, including water, soil, and air. The sample can be processed prior to assay, e.g., to remove cells or cellular debris. The term encompasses samples that have been manipulated after their procurement, such as by treatment with reagents, solubilization, sedimentation, or enrichment for certain components.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used in herein, the terms “identical” or percent “identity”, in the context of describing two or more nucleotide sequences, refer to two or more sequences or subsequences that are the same or have a specified percentage of nucleotides that are the same (for example, a variant nucleotide used in the method of this invention has at least 80% sequence identity, preferably 85%, 90%, 91%, 92%, 93, 94%, 95%, 96%, 97%, 98%, 99%, or 100% identity, to a reference sequence), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids, the BLAST and BLAST 2.0 algorithms and the default parameters discussed below are used.

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium).

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

### Compositions of Probes and Methods of Use

TABLE 1
The sequences of primers, probes, oligonucleotide.

| SEQ ID NO. | Name | Sequence (5′ to 3′) |
|---|---|---|
| 1 | SLC25A13 forward primer | CCAAACCTGTAGGCCGACTCTGCAA |
| 2 | SLC25A13 reverse primer | TTGTGCAACGGGACAATGCTAGGAC |
| 3 | Site 1 mediator strand 1 | ATCGACTTCCGAGACATCATCGACGTCACCGAA |
| | | CTGCGGGTGT |
| 4 | Site 1 mediator strand 2 | GAGTGCGTCCAGGCAGAGCGACTCCATCCGCCC |
| | | CCATGT-C3 spacer |
| 5 | Site 2 mediator strand 1 | GAGTGCGTCCAGGCAGAGCGACTTTTAATTCGC |
| | | TCCTTAA-C3 spacer |
| 6 | Site 2 mediator strand 2 | CAAGTTAGTTTCTCCTATTTTACGACGTCACCGA |
| | | ACTGCGGGTGT-C3 spacer |
| 7 | Site 3 mediator strand 1 | GAGCCAAGGGGACGTATGACCTTAGCAGACATT |
| | | GAACGGCGACGTCACCGAACTGCGGGTGT-C3 |
| | | spacer |
| 8 | Site 3 mediator strand 2 | GAGTGCGTCCAGGCAGAGCGACTCTCTGGAAG |
| | | AGGGAACTCTGCCCTTTAACTTGGCTGAGGCC- |
| | | C3 spacer |
| 9 | Universal F Probe | 6-FAM-AGTCGCTCTGCCTGGACGCACTC-InvdT |
| 10 | Universal Q Probe | ACACCCGCAGTTCGGTGACGTCG-BHQ_2 |
| 11 | HPV 16 forward primer | TACGCACAACCGAAGCGTAG |
| 12 | HPV 16 reverse primer with | ACACCCGCAGTTCGGTGACGTCGACCGGACAGA |
| | tag | GCCCATTACA |
| 13 | HPV 16 mediator strand | GAGTGCGTCCAGGCAGAGCGACTGCCCATTACA |
| | | ATATTGTAACC-C3 spacer |
| 14 | HPV 18 forward primer | TTGGAGTCGTTCCTGTCGTG |
| 15 | HPV 18 reverse primer with | ACACCCGCAGTTCGGTGACGTCGTCACAACATA |
| | tag | GCTGGGCACT |
| 16 | HPV 18 mediator strand | GAGTGCGTCCAGGCAGAGCGACTTAGCTGGGC |
| | | ACTATAGAGGCCA-C3 spacer |
| 17 | HPV 58 forward primer | ACCTCAGATCGCTGCAAAGT |
| 18 | HPV 58 reverse primer with | ACACCCGCAGTTCGGTGACGTCGGTGTCAGGCG |
| | tag | TTGGAGACAT |
| 19 | HPV 58 mediator strand | GAGTGCGTCCAGGCAGAGCGACTTTGGAGACA |
| | | TCTGT-C3-spacer |
| 20 | HPV 33 forward primer | GCATTCCACGCACTGTAGTT |
| 21 | HPV 33 reverse primer with | CTGCCACGATCGACTCGTCTGCAGTCATTGCATG |
| | tag | ATTTGTGCCAAGC |
| 22 | HPV 33 mediator strand | GCTGGACAGTGTGCGGCAGTCACAATTTGTGCC |
| | | AAGCATTGGAGACAAC-C3-spacer |
| 23 | HPV 31 forward primer | CATGCTATGCAACGTCCTGTC |
| 24 | HPV 31 reverse primer with | CTGCCACGATCGACTCGTCTGCAGTGTCAAAGA |
| | tag | CCGTTGTGTCCAG |
| 25 | HPV 31 mediator strand | GCTGGACAGTGTGCGGCAGTCACAGTCCAGAA |
| | | GAAAAACAAAGAC-C3-spacer |
| 26 | HBB forward primer | ACTGACCTCCCACATTOCCT |
| 27 | HBB reverse primer with tag | CTGCCACGATCGACTCGTCTGCAGTGGGCCTTG |
| | | AGCATCTGGATT |
| 28 | HBB mediator strand | GCTGGACAGTGTGCGGCAGTCACAATCTGGATT |
| | | CTGC-C3-spacer |

*The underlined nucleotides indicate locked nucleic acids

The subject invention provides at least 1, 2, 3, 4, 5, or more distinct oligonucleotides labeled with fluorophore (F-Probe), at least 1, 2, 3, 4, 5, 6, or more distinct oligonucleotides labeled with a quencher (Q-Probe), and at least 1, 2, 3, 4, 5, 6, 7, 8, 9, 10 or more distinct oligonucleotides that can hybridize to a target nucleotide sequence (M strand), which can be broadly compatible with the detection of multiple variant locus or multiple target nucleic acid sequences. The sequence of the at least 1 or more M strands is based on the complementarity and stringency of said oligonucleotide to the target nucleotide sequence. Stringency refers to hybridization conditions chosen to optimize binding of polynucleotide sequences with different degrees of complementarity. Stringency is affected by factors such as temperature, salt conditions, the presence of organic solvents in the hybridization mixtures, and the lengths and base compositions of the sequences to be hybridized and the extent of base mismatching, and the combination of parameters is more important than the absolute measure of any one factor. The subject invention further provides at least 1, 2, 3, 4, 5, 6, 7, 8, 9, 10 or more distinct forward primers that can amplify the target nucleotide sequence in conjunction with at least 1, 2, 3, 4, 5, 6, 7, 8, 9, 10 or more distinct reverse primers. In certain embodiments, the reverse primer can be labeled. Preferably, the reverse primer includes the same sequence of the universal Q probe. Through PCR amplification, the amplified targets will be labeled with the universal sequence which is same as the Q probe, thereby reducing the mediator strands that are required in the sensing process. In certain embodiments, the amplification of the target sequence using the forward and reverse primer yields an amplified target nucleotide sequence that comprises a sequence of the forward primer.

In certain embodiments, the F probe can hybridize to the M strand. In certain embodiments, the Q probe can hybridize to the M strand or the sequence of the forward primer within the amplified target nucleotide sequence. In certain embodiments, the 1 or more M strands can hybridize to 1 or more regions of the target nucleotide sequence. In certain embodiments, the F probe and the Q probe can hybridize to the same M strand. In certain embodiments, the F probe and the Q probe can hybridize to distinct M strands, in which each M strand (e.g., M1 strand and M2 strand) hybridizes to a distinct region of the target nucleotide sequence. In certain embodiments, 0, 1, 2, 3, 4, 5, 6, 7, 8, 9, 10, about 15, about 20, about 25, about 30, about 35, about 40, about 45, or about 50 bases separate the regions to which each M strand hybridizes. Most preferably, the separating range is from 2-13 bases.

Typically, the Q probe, the F Probe, and the M strand, can be at least 12 bases, more often about 15, about 18, about 20, about 21, about 22, about 23, about 24, about 25, about 30 bases, or about 60 bases in length. Preferably, the F probe and Q probe should be 15-35 bases, and the M strands should be 25-60 bases. Each M strand is typically designed so that all M strands participating in a particular reaction have a distinct melting temperature, such as, for example, a melting temperature that is greater than or equal to about 5° C. different. In certain embodiments, the melting temperature of the M strand is at least about 30°° C. In certain embodiments, the melting temperature for a set of 6 M strands can be, for example, 31° C., 41° C., 50° C., 58° C., 68° C., and 72° C. In certain embodiments, the melting temperature of the Q probe and the F Probe are about 70° C. to about 80° C. Typically, the Q probe, the F Probe, and the M strand concentrations will range from about 10 nM to about 1 μM. Preferably, for the F and Q probes, the concentration should be about 40 nM-600 nM, and for the M strand, the range should be about 40 nM-200 nM. In certain embodiments, the Q probe, F probe, M strand, forward primer, reverse primer, and target nucleotide sequence can be RNA or DNA. In preferred embodiment, the Q probe, F probe, M strand, forward primer, reverse primer, and target nucleotide sequence are DNA.

Typically, the forward and reverse primers can be at least 12 bases, more often about 15, about 18, about 20, about 21, about 22, about 23, about 24, about 25, about 30, or about 60 bases in length. Preferably, the forward primer is about 15-30 bases, and the reverse primer is about 35-55 bases in length. Primers are typically designed so that all primers participating in a particular reaction have melting temperatures that are within 5° C., and most preferably within 2° C. of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from about 10 nM to about 1 μM. Preferably, the forward primer concentration should be about 80 nM-200 nM. The reverse primer concentration should be about 20 nM-80 nM.

In some embodiments, the Q probe, F probe, and M strand can be used in the detection of multiple mutation loci and multiple nucleic acid molecules. In some embodiments, a sequence can be added to label the target sequence using the forward primer in an asymmetric amplification process. After the asymmetric amplification process, the labeled single-strand amplicons can be detected by the F-Probe and Q-Probe and one M strand for each target. In some embodiments, this can increase the testing capacity by increasing the number of F-Probe and M strands in different fluorescence channels.

In some embodiments, the method of the subject invention uses detection targets. In some embodiments, detection targets are single-strand amplicons that are amplified through asymmetric PCR amplification and other isothermal-based amplification. In some embodiments, the methods of the subject invention include detecting multiple mutation loci (single nucleotide polymorphisms (SNPs), deletion, insertion, or duplication) and multiple nucleic acid molecules, such as, for example, double-stranded DNA, single-stranded DNA, or RNA.

In some embodiments, a signal can be generated as a result of the methods of the subject invention. In some embodiments, the signal generated is a fluorescence signal. For example, in the process of melting curve analysis, different fluorescence data will be obtained at different temperatures. Through the first derivative of fluorescence signal over temperature, one can get the result of the change rate of fluorescence signal at different temperatures. According to those results, one can obtain the melting curve, find its melting temperature value, and judge whether the target object exists. In some embodiments, the fluorescence signal is generated from the melting of F-Probe or Q-Probe.

In certain embodiments, the probes herein can include any useful label, including fluorescent labels and quencher labels at any useful position in the nucleic acid sequence, such as, for example at the 3′-and/or 5′-terminus. Exemplary fluorescent labels include a quantum dot or a fluorophore. Examples of fluorescence labels for use in this method includes fluorescein, 6-FAM™ (Applied Biosystems, Carlsbad, Calif.), TET™ (Applied Biosystems, Carlsbad, Calif.), VIC™ (Applied Biosystems, Carlsbad, Calif), MAX, HEX™ (Applied Biosystems, Carlsbad, Calif), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rhol01, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™M-X (Molecular Probes, Inc., Eugene, Oreg.), Rhodamine 6G, TMR, ABY™ (Applied Biosystems, Carlsbad, Calif.), TAMRA™ (Applied Biosystems, Carlsbad, Calif.), 5-TAMRA, JUN™ (Applied Biosystems, Carlsbad, Calif.), ROX™ (Applied Biosystems, Carlsbad, Calif.), Oregon Green® (Life Technologies, Grand Island, N.Y.), Oregon Green 500, IRDyc® 700 (Li-Cor Biosciences, Lincoln, Nebr.), IRDye 800, WeIIRED D2, WeIIRED D3, WeIIRED D4, and Lightcycler® 640 (Roche Diagnostics GmbH, Mannheim, Germany). In some embodiments, bright fluorophores with extinction coefficients>50,000 M⁻¹ cm⁻¹ and appropriate spectral matching with the fluorescence detection channels can be used.

In certain embodiments, a fluorescently labeled probe is included in a reaction mixture and a fluorescently labeled reaction product is produced. Fluorophores used as labels to generate a fluorescently labeled probe included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumarin 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), cosin and derivatives such as cosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl}amino)naphthalene-1-sulfonic acid), Malachite Green isothiocyanate; 4-methylumbelliferone; orthocresolphthalein; nitrotyrosine; pararosaniline; Phenol Red; B-phycocrytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid; and terbium chelate derivatives.

Exemplary quencher labels include a fluorophore, a quantum dot, a metal nanoparticle, and other related labels. Suitable quenchers include Black Hole Quencher®-1 (Biosearch Technologies, Novato, CA), BHQ-2, Dabcyl, Iowa Black® FQ (Integrated DNA Technologies, Coralville, IA), IowaBlack RQ, QXL™ (AnaSpec, Fremont, CA), QSY 7, QSY 9, QSY 21, QSY 35, IRDye QC, BBQ-650, Atto 540Q, Atto 575Q, Atto 575Q, MGB 3′ CDPI3, and MGB-5′ CDPI3. In one instance, the term “quencher” refers to a substance which reduces emission from a fluorescent donor when in proximity to the donor. In preferred embodiments, the quencher is within 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide bases or within 10 nm of the fluorescent label. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, each of the probes used in a single reaction can have a distinct fluorophore. In certain embodiments, the quencher of each probe can be identical or distinct.

In certain embodiments, the subject methods comprise: providing 1, 2, 3, 4, 5, 6 or more M oligonucleotides (i.e., M strand), which can each bind to a distinct region of the target oligonucleotide sequence; a fluorophore-labeled oligonucleotide, which comprises a region complementary to an M oligonucleotide; a quencher-labeled oligonucleotide, which comprises a region complementary to an M oligonucleotide or a region of the amplified target nucleotide sequence, and, optionally, providing a forward and reverse primer to amplify the target nucleotide sequence as an initial step; mixing the M oligonucleotide, the fluorophore-labeled oligonucleotide, and the quencher-labeled oligonucleotide with the target nucleotide sequence to yield a mixture, wherein the M oligonucleotide hybridizes to the target nucleotide sequence, whereby a quencher of the quencher-labeled oligonucleotide quenches the fluorescence of the fluorophore-labeled oligonucleotide; heating the mixture to about 70° C. to about 80° C., wherein the M oligonucleotide, the fluorophore-labeled oligonucleotide, or the quencher-labeled oligonucleotide releases from the target nucleotide sequence, whereby the fluorophore of the fluorophore-labeled oligonucleotide fluoresces; and measuring the fluorescence of the fluorophore-labeled oligonucleotide. In certain embodiments, the forward primer is provided in an excess concentration relative to the reverse primer, such as, for example, at a concentration at least 2-times, 3-times, or 4-times greater. Preferably, the concentration ratio of forward primer to reverse primer can be 100:1, 50:1, 40:1, 30:1, 20:1, 10:1, 5:1, 4:1, 3:1, or any concentration ratio therebetween.

The subject invention provides universal compositions of melting reporting systems designed to solve issues of high-cost modification and complex background in probe-based melting curve analysis. In certain embodiments, M strands can achieve detection of multiple targets using one set of F-Probe and Q-Probe in single fluorescence channel.

The invention further provides kits, including oligonucleotide probes and primers, packaged into suitable packaging material, optionally in combination with instructions for using the kit components, e.g., instructions for performing a method of the invention. In one embodiment, a kit includes an amount of an oligonucleotides probes and primers, and instructions for running the assay on a label or packaging insert. In further embodiments, a kit includes an article of manufacture, for performing the assay. Preferably, said kit comprises at one primer pair according to the invention. Said kit comprises more than one M strand, e.g. at least two, at least three, at least four, at least five, at least six, at least 7, at least 8, at least 9, or at least 10 different M strands.

In the kit according to the invention, the oligonucleotides (primers, probes) can be either kept separately, or partially mixed, or totally mixed.

In a preferred embodiment, the kit according to the invention contains means for extracting and/or purifying nucleic acid from a biological sample, e.g. from blood, serum, plasma, saliva, or nasal secretions. Such means are well known to those skilled in the art.

In a preferred embodiment, the kit according to the invention contains instructions for the use thereof. Said instructions can advantageously be a leaflet, a card, or the like. Said instructions can also be present under two forms: a detailed one, gathering exhaustive information about the kit and the use thereof, possibly also including literature data; and a quick-guide form or a memo, e.g., in the shape of a card, gathering the essential information needed for the use thereof. Instructions can therefore include instructions for practicing any of the methods of the invention described herein. For example, compositions can be included in a container, pack, or dispenser together with instructions for performing the nucleotide detection assay. Instructions may additionally include storage information, expiration date, or any information required by regulatory agencies such as the Food and Drug Administration or European Medicines Agency for use with a human or animal subject. The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD-or DVD-ROM/RAM, magnetic tape, flash storage, electrical storage media such as RAM and ROM and hybrids of these such as magnetic/optical storage media.

In a preferred embodiment, the kit according to the invention can also contain further reagents suitable for a PCR step, including an asymmetric PCR step.

Such reagents are known to those skilled in the art, and include water, like nuclease-free water, RNase free water, DNAse-free water, PCR-grade water; salts, like magnesium, magnesium chloride, potassium; buffers such as Tris; enzymes, including polymerases, such as Taq, Vent, Pfu (all of them Trade-Marks), activatable polymerase, reverse transcriptase, and the like; nucleotides like deoxynucleotides, dideoxunucleotides, dNTPs, dATP, dTTP, dCTP, dGTP, dUTP; other reagents, like DTT and/or RNase inhibitors; and polynucleotides like polyT, polydT, and other oligonucleotides, e.g., primers.

In a preferred embodiment, said kit is a diagnostics kit, especially an in vitro diagnostics kit

The present invention also relates to the field of diagnostics, prognosis and drug/treatment efficiency monitoring, as above-described.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Melting Sensing Using Universal Compositions of Reporting Probe Systems

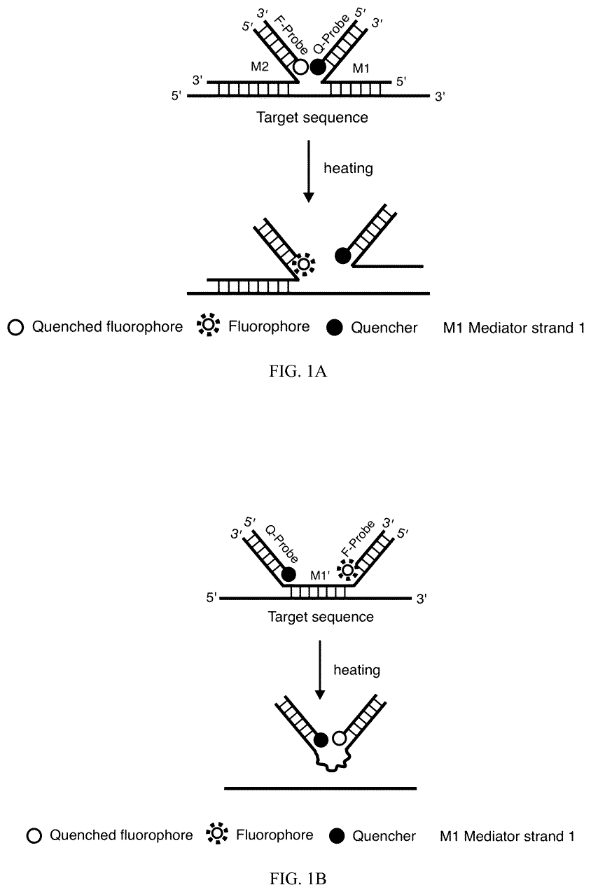

To conduct melting curve analysis, two universal designs of melting reporting systems were provided in this example. In FIG. 1A, the composition is an ‘X-shaped’ structure, which consists of two unmodified mediator oligo strands (M1 strand and M2 strand), respectively, one fluorophore-labeled oligo strand (F-Probe) and one quencher-labeled oligo strand (Q-Probe). The M1 strand and M2 strand both have one part complementary to the target sequence and another part complementary to F-Probe or Q-Probe, respectively. The sequences of F-Probe and Q-Probe are non-target specific universal regions with a melting temperature (Tₘ) around 70-80° C. This high Tₘ design ensures the F-Probe and Q-Probe will keep hybridization status with the M1 and M2 strand during the heating process. During the melting assay, at low temperatures, the fluorescence signal is quenched due to the short distance between F-Probe and Q-Probe (fluorescence resonance energy transfer effect). With the increase of working temperature, when the environment temperature reaches the lower Tₘ of hybrid regions (M1-target and M2-target), the M1 strand or M2 strand will release from the target sequence, thus increasing fluorescence signal. Similarly, the composition can be a ‘Y-shaped’ structure as shown in FIG. 1B, which includes one M1′ strand, one F-Probe, and one Q-Probe. The M1′ strand has one region that can hybridize with target sequences and other regions that can hybridize with F-Probe and Q-Probe in two arms. When M1′ strand hybridizes with the target sequence, there's a certain distance between the F-Probe and Q-Probe thus the fluorescence signal is strong at this moment. Upon the temperature reaching the Tm of the M1′-target hybrid, the M1′ strand with F-Probe and Q-Probe will release from the target sequence. The fluorescence signal will decrease because of the randomly coiled structure of the unhybridized M1′ sequence.

### EXAMPLE 2

### Detection of Multiple Mutation Loci Using Universal Compositions of Melting Reporting Systems

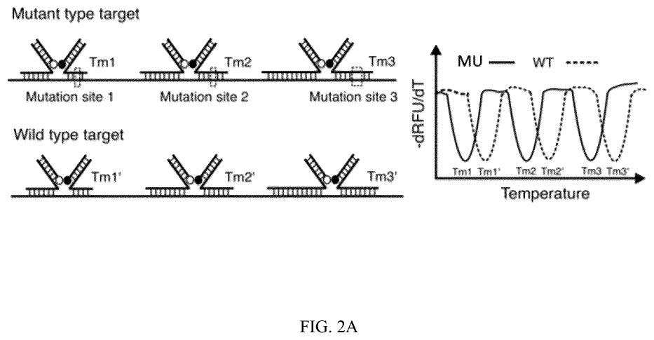

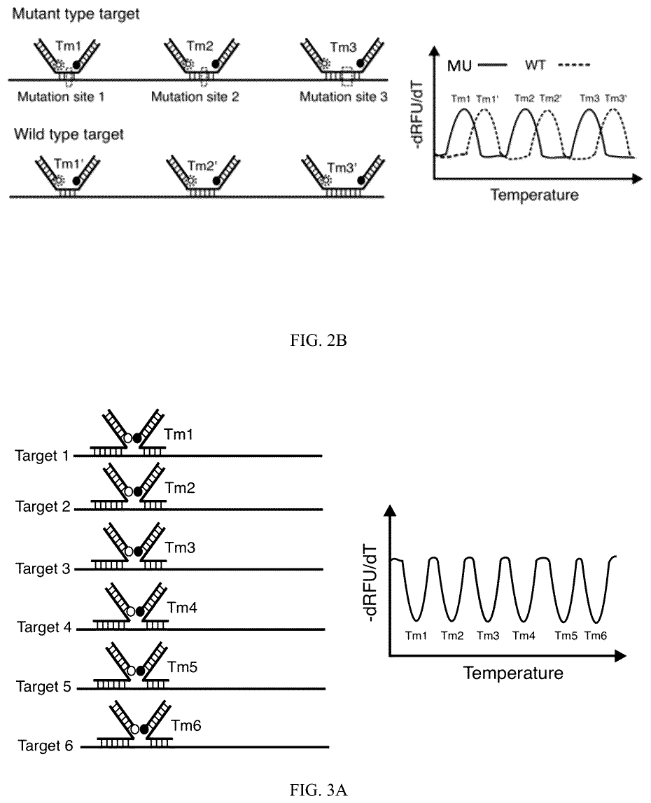

In FIG. 2A, an X-shaped reporting system can be used for the detection of multiple mutation loci in melting curve analysis. X-shaped compositions include two unmodified target-specific oligo strands (M1 strand and M2 strand) and one set of non-target specific universal F-Probe and Q-Probe. Through this design, at least three different mutation loci can be detected in one fluorescence channel using only one set of the fluorophore and quencher-modified probes. These mutation loci include but are not limited to single nucleotide polymorphisms (SNPs), deletion, insertion, duplication, etc. The key differentiation principle is that mismatch or partial hybridization makes the probe melting temperature (Tₘ) lower than that of a perfectly matched probe. Each mutation site will have its specific M1 strand and M2 strand, which are designed to be fully complementary with wild target sequences. For example, if three mutation sites are located on the one genome. three pairs of M strands are used to identify three different mutation sites respectively. Each M stand is comprised of two parts, one is the sequence fully complementary with the F/Q probe for signal readout, another is the sequence that is fully complementary with the target sequences for specific detection of targets. Therefore, the difference among each M strand is the specific target identification sequence. All three pairs of M strands are half complementary with F/Q probes, and half complementary with the target sequences. All of M strands are designed to be half complementary with the wild type sequences, and will hybridize with mutant type sequences with mismatched structures. For sensing the target sequence with three mutation sites, three different pairs of M1 strand and M2 strand and one set of universal F-Probe and Q-Probe will be introduced. One part of the M1 strand and M2 strand will be designed to hybridize with the target site, and another part of the M1 strand and M2 strand will be designed to fully hybridize with the universal F-Probe and Q-Probe. At low temperatures, these X-shaped compositions will hybridize their corresponding target sites, therefore forming multiple M1-target hybrids with different Tₘ values. If the target is a homozygous mutant type with three mutated sites, melting curve analysis will record three downward melting peaks with Tₘ₁, Tₘ₂, and Tₘ₃, respectively. If the target is a homozygous wild type with three mutated sites, the Tₘ of these three melting peaks will shift to T_{m1′}, T_{m2′}, and T_{m3′}, respectively. If the target is a heterozygote, six downward melting peaks will exist in the melting plots with Tₘ₁, T_{m1′}, Tₘ₂, T_{m2′}, Tₘ₃, and T_{m3′}, respectively. Therefore, these three mutation sites can be genotyped according to their Tₘ values of melting peaks. In FIG. 2B, Y-shaped compositions can also be applied in the detection of multiple mutation sites and its fluorescence signal change is opposite to that of an X-shaped structure. For the detection of three mutation sites, each locus will have a target-specific mediator strand (M1′) and one set of universal F-Probe and Q-Probe. After the melting assay, if the target has three mutation sites, three upward melting peaks with the corresponding Tm1, Tm2, and Tm3 will exhibit in the melting plots. If the target doesn't have any of the three mutation sites, three upward melting peaks with the corresponding T_{m1′}, T_{m2′}, and T_{m3′} will exhibit in the melting plots. To test more mutation loci, the testing throughout can be enlarged by introducing multiple sets of universal F-Probe and Q-Probe and multiple pairs of M1 strand and M2 stand in different fluorescence channels.

### EXAMPLE 3

### Detection of Multiple Nucleic Acid Molecules Using Universal Compositions of Melting Reporting Systems

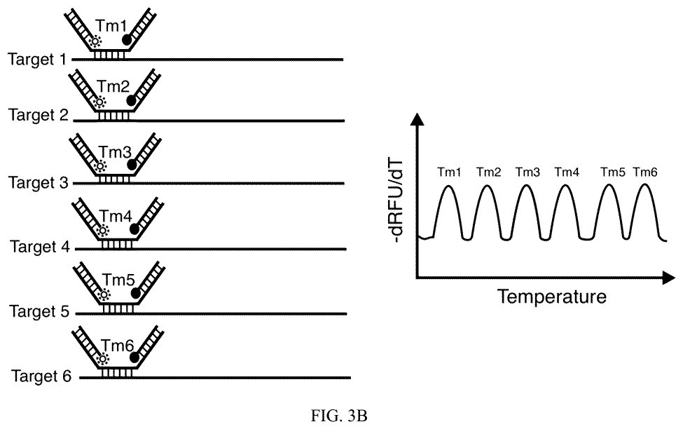

Both X-shaped and Y-shaped compositions can be applied in the detection of multiple nucleic acid molecules. In a single fluorescence channel, at least six subtypes can be analyzed by one set of universal F-Probe and Q-Probe. For the X-shaped probe structure in FIG. 3A, each target sequence will have its corresponding M1 strand and M2 strand. Therefore, for the detection of six samples, six pairs of M1 strand and M2 strand and one set of universal F-Probe and Q-Probe will be required. If all six target sequences exist, the melting plots will have six downward peaks with Tₘ₁, Tₘ₂, Tₘ₃, Tₘ₄, Tₘ₅, and Tₘ₆, respectively. The Tₘ difference between each peak should be designed at least 5° C. for high-resolution differentiation. For the Y-shaped probe structure in FIG. 3B, each target will be assigned one target-specific M1′ strand and one set of universal F-Probe and Q-Probe. If the reaction has six target sequences, after melting curve analysis is performed, there will have six upward melting valleys. If the PCR instrument has six fluorescence channels, the maximum number of subsamples that can be identified in a single reaction is 36.

### EXAMPLE 4

### Detection of The Target Sequence by Asymmetric Labeling Amplification and Universal Compositions of Melting Reporting Systems

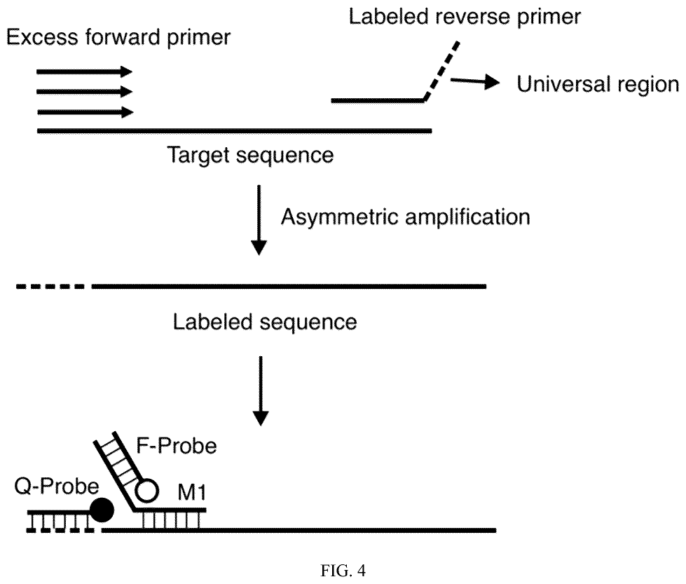

The PCR amplification with melting curve analysis will be integrated to test the target sequence as shown in FIG. 4. First, to generate the single-strand amplicons of the target, asymmetric PCR amplification will be conducted. The amplification forward primer is excess over the reverse primer. A universal region that has the same sequence as Q-Probe will be added to the upstream reverse primer. Therefore, single-strand amplicons labeled with the complementary sequence of the universal region will be generated after asymmetric PCR amplification. To conduct melting sensing, one set of universal F-Probe and Q-Probe and one M1 strand will be incorporated into the reaction. At low temperatures, the M1 strand with F-Probe will hybridize with the target sequence and the Q-Probe will be in hybridization with the labeled universal region of amplicons. The fluorescence intensity is low in this condition due to the short distance between the fluorophore and the quencher. As the increase of environmental temperature during the melting assay, the M1 strand with F-Probe will release from the target sequence and give out strong fluorescence intensity.

### EXAMPLE 5

### Detection of Multiple Nucleic Acid Molecules Using Labeling Strategy and Universal Compositions of Melting Reporting Systems

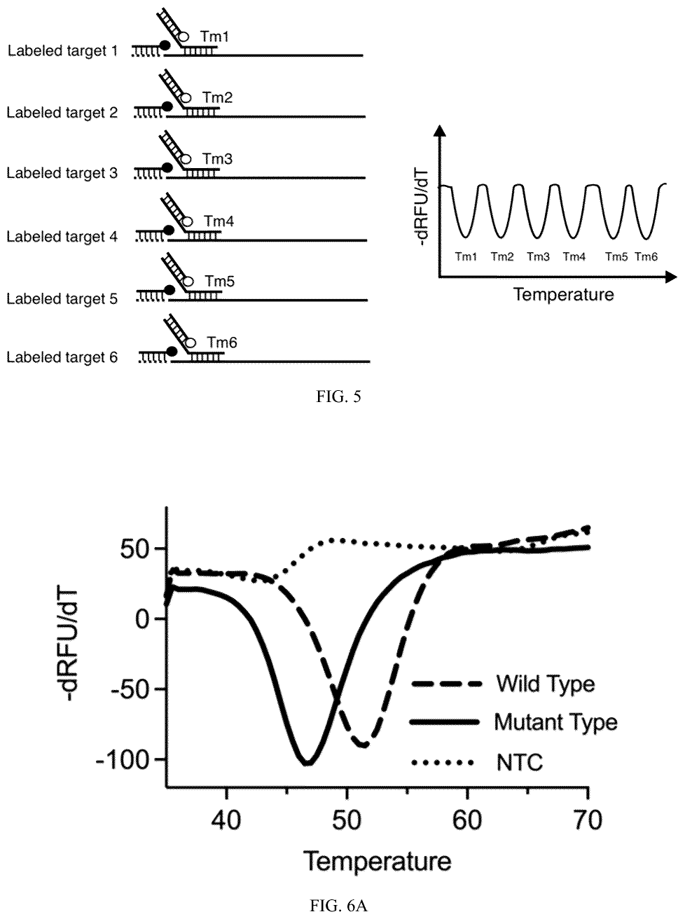

The incorporation of asymmetric labelling amplification can be used to decrease the number of compositions of melting reporting system. Each sample will be first labelled with a universal region that can bind with Q-Probe through asymmetric labelling amplification. The amplified labelled single-strand sequences can be directly used to react with universal compositions of the melting reporting system. One set of universal F-Probe and Q-Probe and six target-specific M1 strands will be introduced to detect six samples in one reaction. The first derivative-d RFU/d T peak of the melting curve analysis can be correlated to the presence of its corresponding target sample in FIG. 5. If all six targets exist, it will have six downward −d RFU/d T peaks. The number of samples tested in the system could be further expanded by increasing M1 strands and F-Probe modified by different fluorophores.

### EXAMPLE 6

### Characterization of X-Shaped and Y-Shaped Compositions Using Homozygous Wild-Type and Mutant-Type Plasmids

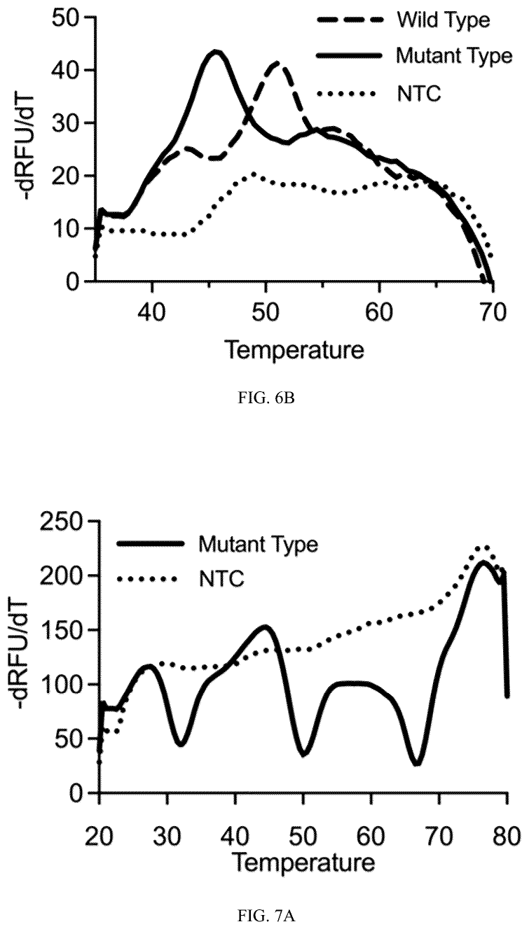

The difference between homozygous wild-type and mutant-type plasmids is one base mutation. The one base mismatch in the target-probe hybrid will result in a Tₘ decrease at around 5-10° C. of the melting valley. For the X-shaped probe system, one M1 strand and M2 strand and one set of F-Probe and Q-Probe were designed to differentiate the wild-type and mutant-type plasmids. Part of the M1 strand and M2 strand were designed fully to be complementary with the wild-type sequence thereby forming a mismatch bubble with the mutant-type plasmids. In FIG. 6A, the results show that X-shaped compositions can differentiate the template with/without single nucleotide polymorphisms (SNPs). The homozygous wild-type displayed an upward melting peak with a Tₘ of 52° C. and homozygous mutant-type plasmids exhibited a 5° C. left shift in melting peak (47° C.). For the Y-shaped probe system, one M1′ strand and one set of F-Probe and Q-Probe were designed to identify the wild-type and mutant-type plasmids. The results in FIG. 6B reveal that the wild-type and mutant-type sequences have distinguishable melting curves with Tₘ values at 52° C. and 47° C., respectively. These experiments demonstrate that both X-shaped and Y-shaped compositions can successfully distinguish target sequences with single base mutation.

### EXAMPLE 7

### Multiplex Detection of Homozygous and Heterozygous Wild-Type and Mutant-Type Plasmids Using X-Shaped Compositions

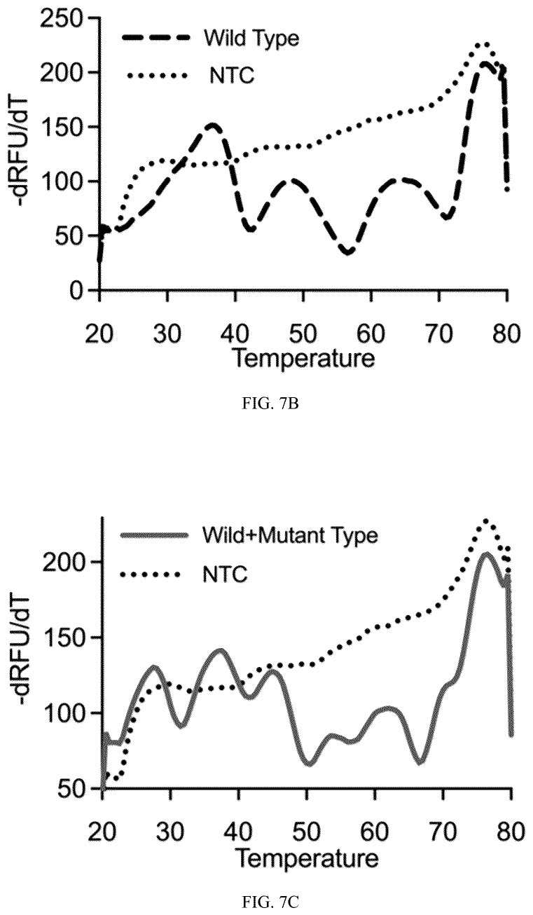

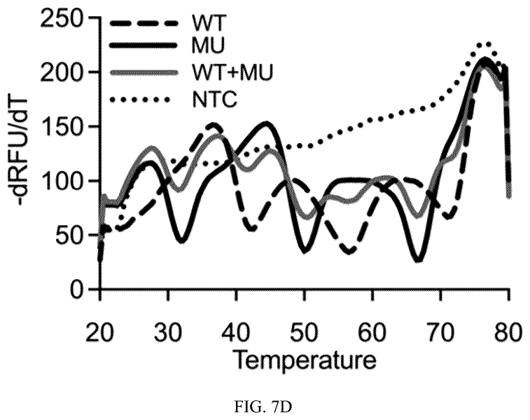

The target sequence has three possible mutation sites to be tested, including two SNP mutation sites and one deletion site. Three X-shaped compositions including one set of universal F-Probe and Q-Probe and three pairs of M1 strand and M2 strand were added to the PCR reaction. The template with three mutation sites, in FIG. 7A, has three melting peaks with Tₘ values at 31° C., 50° C., and 68° C., respectively. For the template without any of the three mutation sites, results in FIG. 7B show that three melting peaks with Tₘ values at 41° C., 58° C., and 72° C., respectively, were observed. FIG. 7C depicts the melting sensing results for heterozygous samples with both wild-type and mutant-type sequences, which have six distinguishable melting peaks with Tₘ values at 31° C., 41° C., 50° C., 58° C., 68° C., and 72° C., respectively. All results combined in FIG. 7D verify that three mutation sites can be differentiated in a single fluorescence channel using only one set of universal F-Probe and Q-Probe.

### EXAMPLE 8

### Multiplex Detection of Five HPV Genotypes Using X-Shaped Compositions

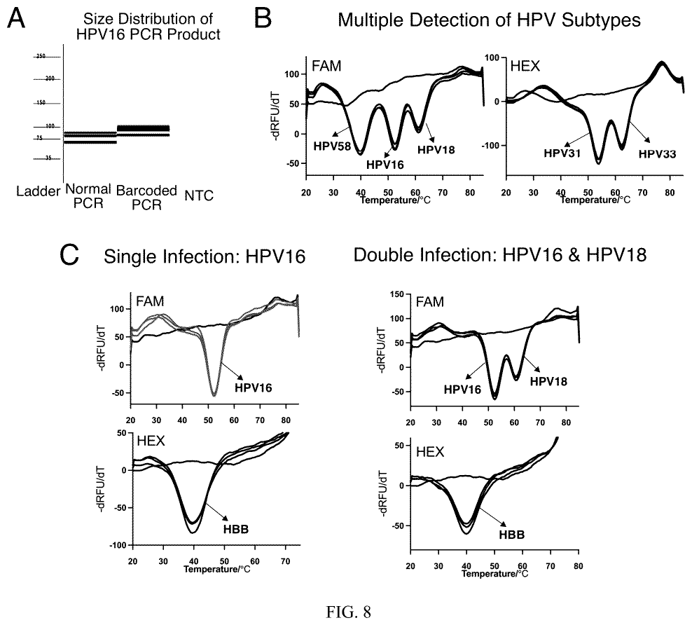

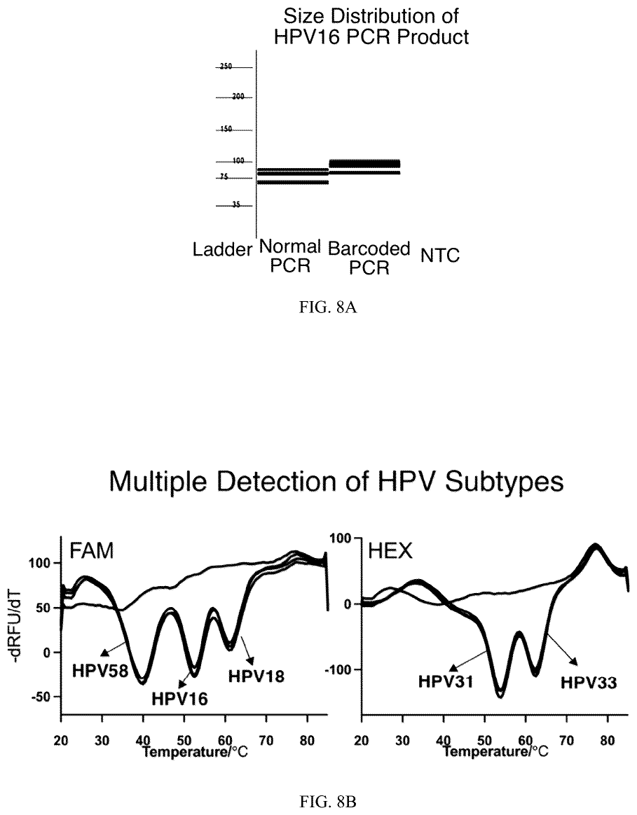

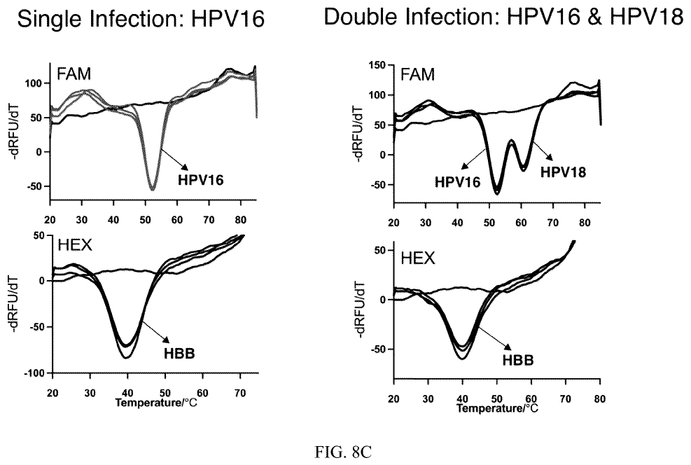

The barcoding amplification and X-shaped compositions were incorporated for the detection of HPV subtypes. To check the feasibility of PCR barcoding amplification, the amplified amplicons from normal and barcoding PCR were analyzed by fragment analyzer. The size distribution results in FIG. 8A show the products from barcoding PCR are longer than that of normal PCR, which indicates that the barcode sequence in the reverse primer has been successfully added to the target sequence. Next, HPV subtype 16, 18, 58, 33, and 31 were analysed in two fluorescent channels FAM and HEX. The melting curve results in FIG. 8B show that all five subtypes can be correctly identified in FAM and HEX channels. To test the cross-reactivity among different subtypes, double and single infection experiments with HBB reference genes were performed to verify it. The results demonstrate that no cross-reactivity was observed in FIG. 8C.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Y. Zou, Q. qiang Dai, W. jing Tao, X. ling Wen, D. feng Fong, H. Deng, W. ping Zhou, M. Li, L. Zhang, Suspension array-based deafness genetic screening in 53,033 Chinese newborns identifies high prevalence of 109 G>A in GJB2, Int. J. Pediatr. Otorhinolaryngol. 126 (2019). https://doi.org/10.1016/j.ijporl.2019.109630.
[2] H. Duan, N. Liu, Z. Zhao, Y. Liu, Y. Wang, Z. Li, M. Xu, D.S. Cram, X. Kong, Non-invasive prenatal testing of pregnancies at risk for phenylketonuria, Arch. Dis. Child. Fetal Neonatal Ed. 104 (2019) F24-F29. https://doi.org/10.1136/archdischild-2017-313929.
[3] W.X. Lin, H.S. Zeng, Z.H. Zhang, M. Mao, Q.Q. Zheng, S.T. Zhao, Y. Cheng, F.P. Chen, W.R. Wen, Y.Z. Song, Molecular diagnosis of pediatric patients with citrin deficiency in China: SLC25A13 mutation spectrum and the geographic distribution, Sci. Rep. 6 (2016) 1-12. https://doi.org/10.1038/srep29732.
[4] J.T. Robinson, H. Thorvaldsdóttir, A.M. Wenger, A. Zehir, J.P. Mesirov, Variant review with the integrative genomics viewer, Cancer Res. 77 (2017) e31-e34. https://doi.org/10.1158/0008-5472.CAN-17-0337.
[5] E.M. Burd, Human papillomavirus and cervical cancer, Clin. Microbiol. Rev. 16 (2003) 1-17. https://doi.org/10.1128/CMR.16.1.1-17.2003.
[6] J.Y. Shang, G.J. Zhang, S. Liao, Y. Huang, W.J. Yu, W. He, G.Y. Yang, T.Y. Li, X.W. Chen, B. Zhong, Q. Wang, Q. Wang, R.R. Li, H. Wang, A multiplex PCR for differential detection of Echinococcus granulosus sensu stricto, Echinococcus multilocularis and Echinococcus canadensis in China, Infect. Dis. Poverty. 8 (2019). https://doi.org/10.1186/S40249-019-0580-2.
[7] G. Scuderi, A.F. Catara, G. Licciardello, Genotyping Citrus tristeza virus Isolates by Sequential Multiplex RT-PCR and Microarray Hybridization in a Lab-on-Chip Device, 2019. https://doi.org/10.1007/978-1-4939-9558-5_9.
[8] C.A. Tabuloc, K.M. Lewald, W.R. Conner, Y. Lec, E.K. Lec, A.B. Cain, K.E. Godfrey, J. Arnó, N. Agustí, C.R. Perini, J.C. Guedes, F.G. Zalom, J.C. Chiu, Sequencing of Tuta absoluta genome to develop SNP genotyping assays for species identification, J. Pest Sci. (2004). 92 (2019) 1397-1407. https://doi.org/10.1007/s10340-019-01116-6.
[9] R.J. Dikdan, S.A.E. Marras, A.P. Field, A. Brownlee, A. Cironi, D.A. Hill, S. Tyagi, Multiplex PCR Assays for Identifying all Major Severe Acute Respiratory Syndrome Coronavirus 2 Variants, J. Mol. Diagnostics. 24 (2022) 309-319. https://doi.org/10.1016/j.jmoldx.2022.01.004.
[10] D. Hur, M.S. Kim, M. Song, J. Jung, H. Park, Detection of genetic variation using dual-labeled peptide nucleic acid (PNA) probe-based melting point analysis, Biol. Proced. Online. 17 (2015) 1-12. https://doi.org/10.1186/s12575-015-0027-5.
[10] G. Feng, W. Han, J. Shi, R. Xia, J. Xu, Analysis of the application of a gene chip method for detecting Mycobacterium tuberculosis drug resistance in clinical specimens: a retrospective study, Sci. Rep. 11 (2021) 1-11. https://doi.org/10.1038/s41598-021-97559-y.
[12] G. Russo, C. Zegar, A. Giordano, Advantages and limitations of microarray technology in human cancer, Oncogene. 22 (2003) 6497-6507. https://doi.org/10.1038/sj.onc.1206865.
[13] M. Gulilat, T. Lamb, W.A. Teft, J. Wang, J.S. Dron, J.F. Robinson, R.G. Tirona, R.A. Hegele, R.B. Kim, U.I. Schwarz, Targeted next generation sequencing as a tool for precision medicine, BMC Med. Genomics. 12 (2019) 1-17. https://doi.org/10.1186/s12920-019-0527-2.
[14] M. Hong, S. Tao, L. Zhang, L.T. Diao, X. Huang, S. Huang, S.J. Xic, Z.D. Xiao, H. Zhang, RNA sequencing: new technologies and applications in cancer research, J. Hematol. Oncol. 13 (2020) 1-16. https://doi.org/10.1186/s13045-020-01005-x.
[15] H. Wang, J.A. Miller, M. Verghese, M. Sibai, D. Solis, K.O. Mfuh, B. Jiang, N. Iwai, M. Mar, C.H. Huang, F. Yamamoto, M.K. Sahoo, J. Zehnder, B.A. Pinsky, Multiplex sars-cov-2 genotyping reverse transcriptase per for population-level variant screening and epidemiologic surveillance, J. Clin. Microbiol. 59 (2021). https://doi.org/10.1128/JCM.00859-21.
[16] A. Vaupel, B. Hommel, L. Beule, High-resolution melting (HRM) curve analysis as a potential tool for the identification of earthworm species and haplotypes, PeerJ. 10 (2022). https://doi.org/10.7717/peerj.13661.
[17] Q. Huang, Z. Liu, Y. Liao, X. Chen, Y. Zhang, Q. Li, Multiplex fluorescence melting curve analysis for mutation detection with dual-labeled, self-quenched probes, PLOS One. 6 (2011). https://doi.org/10.1371/journal.pone.0019206.
[18] S. Liao, L. Wang, X. Ji, J. Chen, Q. Li, L. Ma, Simultaneous detection of 15 respiratory pathogens with a fluorescence probe melting curve analysis-based multiplex real-time PCR assay., Int. J. Mol. Epidemiol. Genet. 10 (2019) 29-37. http://www.ncbi.nlm.nih.gov/pubmed/31149327%0Ahttp://www.pubmedcentral.nih.gov/articlerender.fcgi?artid=PMC6526376.
[19] W. Yu, J. Yao, Z. Zhang, Simultaneous Detection of Three Genotypes of Gene Methylene Tetrahydrofolate Reductase and Methionine Synthase Reductase Based on Multiplex Asymmetric Real-Time PCR-HRM Biosensing, (2022). https://doi.org/10.1021/acs.analchem.2c02096.
[20] X. Zhuang, X. Lu, H.L.L. Yu, I.-M. Hsing, Unique Barcoded Primer-Assisted Sample-Specific Pooled Testing (Uni-Pool) for Large-Scale Screening of Viral Pathogens, Anal. Chem. (2022) acs.analchem.1c05204. https://doi.org/10.1021/ACS.ANALCHEM.1C05204.
[21] Q. Huang, D. Chen, C. Du, Q. Liu, S. Lin, L. Liang, Y. Xu, Y. Liao, Q. Li, Highly multiplex PCR assays by coupling the 5′-flap endonuclease activity of Taq DNA polymerase and molecular beacon Proc. Natl. Acad. reporters, Sci. 119 (2022). https://doi.org/10.1073/PNAS.2110672119.
[22] A. Markou, E. Tzanikou, I. Ladas, G.M. Makrigiorgos, E. Lianidou, Nuclease-Assisted Minor Allele Enrichment Using Overlapping Probes-Assisted Amplification-Refractory Mutation System: An Approach for the Improvement of Amplification-Refractory Mutation System-Polymerase Chain Reaction Specificity in Liquid Biopsies, Anal. Chem. 91 (2019) 13105. https://doi.org/10.1021/ACS.ANALCHEM.9B03325.
[23] J. Tanaka, T. Nakagawa, K. Harada, C. Morizane, H. Tanaka, S. Shiba, A. Ohba, S. Hijioka, E. Takai, S. Yachida, Y. Kamura, T. Ishida, T. Yokoi, C. Uematsu, Efficient and accurate KRAS genotyping using digital PCR combined with melting curve analysis for ctDNA from pancreatic cancer patients, Sci. Rep. 13 (2023) 3039. https://doi.org/10.1038/s41598-023-30131-y.

We claim:

1. A method for multiplex genotyping of a target nucleotide sequence, the method comprising:
i) providing an Ml oligonucleotide, an M2 oligonucleotide, a fluorophore-labeled oligonucleotide, and a quencher-labeled oligonucleotide, wherein the M1 oligonucleotide and the M2 oligonucleotide comprise a nucleotide sequence complementary to a target nucleotide sequence, the M1 oligonucleotide comprises a nucleotide sequence complementary to the quencher-labeled oligonucleotide, and the M2 oligonucleotide comprises a nucleotide sequence complementary to the fluorophore-labeled oligonucleotide;
ii) mixing the M1 oligonucleotide, the M2 oligonucleotide, the fluorophore-labeled oligonucleotide, and the quencher-labeled oligonucleotide with the target nucleotide sequence to yield a mixture, wherein the M1 oligonucleotide hybridizes to the target nucleotide sequence and the M2 oligonucleotide hybridizes to target nucleotide sequence in close proximity, whereby a quencher of the quencher-labeled oligonucleotide quenches the fluorescence of the fluorophore-labeled oligonucleotide;
iii) heating the mixture to about 70°° C. to about 80° C., wherein the M1 oligonucleotide or the M2 oligonucleotide releases from the target nucleotide sequence, whereby the fluorophore of the fluorophore-labeled oligonucleotide fluoresces; and
iv) measuring the fluorescence of the fluorophore-labeled oligonucleotide.

2. The method of claim 1, wherein the M1 oligonucleotide is about 25 to about 60 bases long, the nucleotide sequence complementary to the quencher-labeled oligonucleotide of the M1 oligonucleotide is about 5 to about 50 bases, and the nucleotide sequence complementary to the target nucleotide sequence of the M1 oligonucleotide is about 5 to about 50 bases long.

3. The method of claim 1, wherein the nucleotide sequence complementary to the target nucleotide sequence of the M1 oligonucleotide is about 40% to 100% complementary to the target nucleotide sequence.

4. The method of claim 1, wherein the M2 oligonucleotide is about 25 to about 60 bases long, the nucleotide sequence complementary to the fluorophore-labeled oligonucleotide of the M2 oligonucleotide is about 5 to about 50 bases, and the nucleotide sequence complementary to the target nucleotide sequence of the M2 oligonucleotide is about 5 to about 50 bases long.

5. The method of claim 1, wherein the nucleotide sequence complementary to the target nucleotide sequence of the M2 oligonucleotide is about 40% to 100% complementary to the target nucleotide sequence.

6. The method of claim 1, wherein the fluorophore-labeled oligonucleotide is about 15 to about 35 bases long and the quencher-labeled oligonucleotide is about 15 to about 35 bases long.

7. The method of claim 1, wherein each M1 oligonucleotide and each M2 oligonucleotide has a melting temperature between about 30° C. to about 80° C.

8. The method of claim 1, comprising
i) providing a plurality of M1 oligonucleotides, a plurality of M2 oligonucleotides, a fluorophore-labeled oligonucleotide, and a quencher-labeled oligonucleotide, wherein each M1 oligonucleotide and each M2 oligonucleotide targets a distinct variation within the target nucleotide sequence, each M1 oligonucleotide comprises a nucleotide sequence complementary to the quencher-labeled oligonucleotide, and each M2 oligonucleotide comprises a nucleotide sequence complementary to the fluorophore-labeled oligonucleotide;
ii) mixing the plurality of M1 oligonucleotides, the plurality of M2 oligonucleotides, the fluorophore-labeled oligonucleotide, and the quencher-labeled oligonucleotide with the target nucleotide sequence to yield a mixture, wherein each M1 oligonucleotide hybridizes to the target nucleotide sequence and each M2 oligonucleotide hybridizes to target nucleotide sequence in close proximity, whereby a quencher of the quencher-labeled oligonucleotide quenches the fluorescence of the fluorophore-labeled oligonucleotide;
iii) heating the mixture to about 70° C. to about 80° C., wherein each M1 oligonucleotide or each M2 oligonucleotide releases from the target nucleotide sequence, whereby the fluorophore of the fluorophore-labeled oligonucleotide fluoresces; and
iv) measuring the fluorescence of the fluorophore-labeled oligonucleotide.

9. The method of claim 8, wherein each M1 oligonucleotide and each M2 oligonucleotide has a melting temperature between about 30° C. to about 80° C.

10. A method for multiplex genotyping of a target nucleotide sequence, the method comprising:
i) providing an M1′ oligonucleotide, a fluorophore-labeled oligonucleotide, and a quencher-labeled oligonucleotide, wherein the M1′ oligonucleotide comprises a nucleotide sequence complementary to the target nucleotide sequence, the M1′ oligonucleotide comprises a nucleotide sequence complementary to the quencher-labeled oligonucleotide, and the M1′ oligonucleotide comprises a nucleotide sequence complementary to the fluorophore-labeled oligonucleotide;
ii) mixing the M1′ oligonucleotide, the fluorophore-labeled oligonucleotide, and the quencher-labeled oligonucleotide with the target nucleotide sequence to yield a mixture, wherein the M1′ oligonucleotide hybridizes to the target nucleotide sequence, whereby the quencher of the quencher labeled oligonucleotide quenches the fluorescence of a fluorophore-labeled oligonucleotide;
iii) heating the mixture to about 70° C. to about 80° C., wherein the M1′ oligonucleotide releases from the target nucleotide sequence, whereby the fluorophore of the fluorophore-labeled oligonucleotide fluoresces; and
iv) measuring the fluorescence of the fluorophore-labeled oligonucleotide.

11. The method of claim 10, wherein the M1′ oligonucleotide is about 25 to about 60 bases long, the nucleotide sequence complementary to the quencher-labeled oligonucleotide of the M1′ oligonucleotide is about 5 to about 50 bases, and the nucleotide sequence complementary to the target nucleotide sequence of the M1′ oligonucleotide is about 5 to about 50 bases long.

12. The method of claim 10, wherein the nucleotide sequence complementary to the target nucleotide sequence of the M1′ oligonucleotide is about 40% to 100% complementary to the target nucleotide sequence.

13. The method of claim 10, wherein the fluorophore-labeled oligonucleotide is about 15 to about 35 bases long and the quencher-labeled oligonucleotide is about 15 to about 35 bases long.

14. The method of claim 10, wherein the M1′ oligonucleotide has a melting temperature between about 30° C. to about 80° C.

15. The method of claim 10, comprising
i) providing a plurality of M1′ oligonucleotides, a fluorophore-labeled oligonucleotide, and a quencher-labeled oligonucleotide, wherein each M1′ oligonucleotide targets a distinct variation within the target nucleotide sequence, each M1′ oligonucleotide comprises a nucleotide sequence complementary to the quencher-labeled oligonucleotide, and each M1′ oligonucleotide comprises a nucleotide sequence complementary to the fluorophore-labeled oligonucleotide;
ii) mixing the plurality of M1′ oligonucleotides, the fluorophore-labeled oligonucleotide, and the quencher-labeled oligonucleotide with the target nucleotide sequence to yield a mixture, wherein each M1′ oligonucleotide hybridizes to the target nucleotide sequence, whereby a quencher of the quencher-labeled oligonucleotide quenches the fluorescence of the fluorophore-labeled oligonucleotide;
iii) heating the mixture to about 70° C. to about 80° C., wherein each M1′ oligonucleotide releases from the target nucleotide sequence, whereby the fluorophore of the fluorophore-labeled oligonucleotide fluoresces; and
iv) measuring the fluorescence of the fluorophore-labeled oligonucleotide.

16. The method of claim 15, wherein each M1′ oligonucleotide has a melting temperature between about 30° C. to about 80° C.

17. A method for multiplex genotyping of a target nucleotide sequence, the method comprising:
i) amplifying the target nucleotide sequence with a forward primer and a reverse primer to yield an amplified target nucleotide sequence comprising a sequence of the forward primer; and the reverse primer;
ii) providing an M1 oligonucleotide, a fluorophore-labeled oligonucleotide, and a quencher-labeled oligonucleotide, wherein the M1 oligonucleotide comprises a nucleotide sequence complementary to the target nucleotide sequence, the quencher-labeled oligonucleotide comprises a nucleotide sequence complementary to the reverse primer sequence of the amplified target nucleotide sequence, and the M1 oligonucleotide comprises a nucleotide sequence complementary to the fluorophore-labeled oligonucleotide;
iii) mixing the M1 oligonucleotide, the fluorophore-labeled oligonucleotide, and the quencher-labeled oligonucleotide with the amplified target nucleotide sequence to yield a mixture, wherein the M1 oligonucleotide hybridizes to the target nucleotide sequence and the fluorophore-labeled oligonucleotide, and the quencher-labeled oligonucleotide hybridizes to the reverse primer sequence of the amplified target nucleotide sequence, whereby a quencher of the quencher-labeled oligonucleotide quenches the fluorescence of the fluorophore-labeled oligonucleotide;
iv) heating the mixture to about 70° C. to about 80° C., wherein the M1 oligonucleotide or the quencher-labeled oligonucleotide releases from the target nucleotide sequence, whereby the fluorophore of the fluorophore-labeled oligonucleotide fluoresces; and
v) measuring the fluorescence of the fluorophore-labeled oligonucleotide.

18. The method of claim 17, wherein the M1 oligonucleotide is about 25 to about 60 bases long, the nucleotide sequence complementary to the fluorophore-labeled oligonucleotide of the M1 oligonucleotide is about 5 to about 50 bases, and the nucleotide sequence complementary to the target nucleotide sequence of the M1 oligonucleotide is about 5 to about 50 bases long.

19. The method of claim 17, wherein the nucleotide sequence complementary to the target nucleotide sequence of the M1 oligonucleotide is about 40% to 100% complementary to the target nucleotide sequence.

20. The method of claim 17, wherein the fluorophore-labeled oligonucleotide is about 15 to about 35 bases long and the quencher-labeled oligonucleotide is about 15 to about 35 bases long and the nucleotide sequence complementary to the target nucleotide sequence of the quencher-labeled oligonucleotide is about 5 to about 50 bases long.

21. The method of claim 17, wherein the M1 oligonucleotide and the quencher-labeled oligonucleotide has a melting temperature between about 30° C. to about 80° C.

22. The method of claim 17, comprising
i) amplifying the target nucleotide sequence with a forward primer and a reverse primer to yield an amplified target nucleotide sequence comprising a sequence of the forward primer; and the reverse primer;
ii) providing a plurality of M1 oligonucleotides, a fluorophore-labeled oligonucleotide, and a quencher-labeled oligonucleotide, wherein each M1 oligonucleotide targets a distinct variation within the target nucleotide sequence, the quencher-labeled oligonucleotide comprises a nucleotide sequence complementary to the reverse primer sequence of the amplified target nucleotide sequence, and the M1 oligonucleotide comprises a nucleotide sequence complementary to the fluorophore-labeled oligonucleotide;
iii) mixing the plurality of MI oligonucleotides, the fluorophore-labeled oligonucleotide, and the quencher-labeled oligonucleotide with the amplified target nucleotide sequence to yield a mixture, wherein each M1 oligonucleotide hybridizes to the target nucleotide sequence and the fluorophore-labeled oligonucleotide, and the quencher-labeled oligonucleotide hybridizes to the reverse primer sequence of the amplified target nucleotide sequence, whereby a quencher of the quencher-labeled oligonucleotide quenches the fluorescence of the fluorophore-labeled oligonucleotide;
iv) heating the mixture to about 70° C. to about 80° C., wherein each M1 oligonucleotide or the quencher-labeled oligonucleotide releases from the target nucleotide sequence, whereby the fluorophore of the fluorophore-labeled oligonucleotide fluoresces; and
v) measuring the fluorescence of the fluorophore-labeled oligonucleotide.

23. The method of claim 17, wherein each M1 oligonucleotide and the quencher-labeled oligonucleotide has a melting temperature between about 30°° C. to about 80° C.
